(19) United States
(12) Patent Application Publication
(10) Pub. No.: US 2021/0024785 A1
(43) Pub. Date: Jan. 28, 2021

(54) DETACHABLE ADHESIVE COMPOSITION SYSTEMS AND METHODS

(71) Applicant: PIMEA 6, Kefar Saba (IL)

(72) Inventor: Ron NAGAR, Tel Aviv (IL)

(21) Appl. No.: 16/980,273

(22) PCT Filed: Mar. 14, 2019
(86) PCT No.: PCT/IB2019/052088

§ 371 Date: Sep. 11, 2020

Related U.S. Application Data
(60) Provisional application No. 62/642,650, filed on Mar. 14, 2018.
(60) Provisional application No. 62/659,208, filed on Apr. 18, 2018.

(51) Int. Cl. C09J 11/08; C09J 5/00; C09D 7/65; C09D 7/40; C04B 24/24; C04B 20/00; A61K 8/11; A61Q 3/02; A61K 6/30

(52) U.S. Cl. CPC C09J 11/08; C09J 5/00; C09D 7/65; C09D 7/70; C04B 24/24; C04B 20/002; C04B 2103/0073; A61Q 3/02; A61K 6/30; C09J 2301/416; C09J 2301/502; C09J 2301/412; C09J 2301/408; A61K 8/11

(57) ABSTRACT

A detachable adhesive composition for selectively adhering to an object and detaching therefrom, including an adhesive compound and a plurality of selectively activated particles. The composition is configured to adhere to an object when the selectively activated particles are inactive, and the plurality of selectively activated particles are configured to be activated upon exposure to and absorption of energy causing the selectively activated particles to flow and expand so as to form micro-cracks in the adhesive compound, flow into the micro-cracks, and react with the adhesive compound by at least one of at least partially chemically reacting with adhesive compound so as to at least partially digest the adhesive compound, and lubricating the adhesive compound so as to allow the detachable adhesive composition to detach from the object.

### CROSS-REFERENCE TO RELATED APPLICATIONS

This application claims priority to U.S. Provisional Patent Application No. 62/642,650, filed Mar. 14, 2018, and entitled “Cement Compositions Methods of Making and Using and Devices for Use with the Same”; and U.S. Provisional Patent Application No. 62/659,208, filed Apr. 18, 2018, and entitled “Adhesive Compositions Methods of Making and Using and Devices for Use with the Same,” the disclosures of which are hereby expressly incorporated by reference in their entireties.

### TECHNICAL FIELD

Embodiments of the present disclosure generally relate to detachable adhesive compositions, methods and systems using the detachable adhesive compositions and methods for processing the detachable adhesive compositions.

### BACKGROUND

Adhesives are widely used to adhere articles to objects or for filling cavities or gaps in various objects. In some applications it is desirable to remove the article from the object while ensuring the article or object remain intact and without use of heavy mechanical force.

### SUMMARY OF SOME OF THE EMBODIMENTS

There is provided according to an embodiment of the present disclosure, a detachable adhesive composition for selectively adhering to an object and detaching therefrom, including an adhesive compound and a plurality of selectively activated particles, wherein the composition is configured to adhere to an object when the selectively activated particles are inactive, and the plurality of selectively activated particles are configured to be activated upon exposure to and absorption of energy causing the selectively activated particles to flow and expand so as to form micro-cracks in the adhesive compound, flow into the micro-cracks, and react with the adhesive compound by at least one of: at least partially chemically reacting with adhesive compound so as to at least partially digest the adhesive compound, and lubricating the adhesive compound so as to allow the detachable adhesive composition to detach from the object.

In some embodiments, the selectively activated particles are selectively flowable. In some embodiments, the composition selectively adheres an article to the object. In some embodiments, the composition is configured for selectively adhering to the object for filling cavities within the object. In some embodiments, the article includes at least one of a dental crown, bracket, bridge or brace, and the object includes any one of a tooth, dental implant or abutment. In some embodiments, the composition forms a gel nail polish, and the object includes a human nail. In some embodiments, the article includes at least one of a tile or flooring, and the object includes any one of a floor, wall or ceiling.

In some embodiments, the plurality of selectively activated particles include a weight or volume of 1% to 10% of a total weight or volume of the composition.

In some embodiments, the adhesive compound is configured with a first level of a reacting parameter (RP) the selectively activated particles are configured with a second level of RP, and the difference between the first level the second level is configured to be at a first predetermined value, whereupon the selectively activated particles are at least partially inactive and thereby nonreactive with the adhesive compound, and at a second predetermined value whereupon the selectively activated particles are activated. In some embodiments, the RP includes a pH level. In some embodiments, each level of RP is measured by RP units and the second predetermined value is greater than an RP unit of 5. In some embodiments, wherein the adhesive compound includes an original pH of less than 7 and the plurality of selectively activated particles includes an original pH of greater than 7.

In some embodiments, the adhesive compound includes at least one of a cement, glue or binder. In some embodiments, the selectively activated particles include a phase change material.

In some embodiments, the selectively activated particles include granules each having a diameter of less than 300 microns. In some embodiments, the plurality of selectively activated particles include granules each having a diameter of less than 100 microns. In some embodiments, the plurality of selectively activated particles include granules each having a diameter of less than 50 microns. In some embodiments, the plurality of selectively activated particles include an un-encapsulated phase change material.

In some embodiments, the plurality of selectively activated particles include microencapsulated water. In some embodiments, the plurality of selectively activated particles include a microcapsule including an encapsulated material, and the microcapsule is formed of a material configured to burst upon the application of the energy to facilitate the flow of the encapsulated material into the adhesive compound.

In some embodiments, the adhesive compound includes a cement compound and a hardening mixture configured to harden the cement compound for adhering to the object. In some embodiments, the plurality of selectively activated particles include a phase change material (PCM) including at least one of an inorganic PCM including at least one of salt hydrates, salts, metals and alloys, and an organic PCM including at least one of paraffin, fatty acids, oils, biocompatible oils, vegetable oils, alcohols and glycols.

In some embodiments, the adhesive compound is selected from the group consisting of cement mixtures, cement compounds, glue compounds, glue mixtures, bond compounds, bond mixtures, resin compounds, resin mixtures, epoxies, cyanoacrylates, spray adhesives, pressure sensitive adhesives, fabric adhesives, hot glue, yellow wood glue, polyurethane adhesive, synthetic polymer-including adhesives, synthetic monomer-including adhesives, solvent—including adhesives, plant based adhesives, animal based adhesives, mineral based adhesives, Portland Cement, sealers and bioceremic (BC) sealers.

In some embodiments, the adhesive compound is formed of a plurality of particulates, and the size of particles of the plurality of selectively activated particles is equal to or less than the size of the plurality of particulates of the adhesive compound. In some embodiments, the adhesive compound is formed of a plurality of particulates, and the size of each of the plurality of selectively activated particles is at least 150% the size of each of the plurality of particulates of the adhesive compound.

In some embodiments, the energy includes energy from at least one of a heat-operated device, a sonically-operated device, an electromagnetically-operated device, a UV-operated device, an optically-operated device, a microwave-operated device, and a radiation-operated device. In some embodiments, the composition is configured to be reversible by at least partially reversing the reaction between the plurality of selectively activated particles and the adhesive compound, so as to render the composition reusable.

In some embodiments, the plurality of selectively activated particles are uniformly dispersed within the composition, such that a 1:1 (one to one) ratio of the increase of volume of the composition to a resultant increase in the amount of the plurality of selectively activated particles. In some embodiments, the composition is configured to discharge one or more byproducts and the plurality of selectively activated particles are configured, when at least partially inactive, to be nonreactive with the one or more byproducts. In some embodiments, the plurality of selectively activated particles include a reagent for at least partially digesting the adhesive compound. In some embodiments, the plurality of selectively activated particles include a lubricant for lubricating the adhesive compound.

There is provided according to an embodiment of the present disclosure, a selective adhesion method for selectively adhering a detachable adhesive composition to an object and detaching therefrom, including forming the detachable adhesive composition by mixing an adhesive compound with a plurality of selectively activated particles, adhering the composition to the object when the plurality of selectively activated particles are at least partially inactive and nonreactive with the adhesive compound, and exposing the composition to energy so as to cause the plurality of selectively activated particles to expand and form micro-cracks in the adhesive compound, flow into the micro-cracks, and react with the adhesive compound in at least one of the flowing reactions: chemically reacting the plurality of selectively activated particles with the adhesive compound resulting in at least a partial digestion of the adhesive compound, and lubricating the adhesive compound, detach the composition from the object.

There is provided according to an embodiment of the present disclosure, a detachable adhesive system for selectively adhering an article to an object and detaching it therefrom, including a detachable adhesive composition and a griping element for gripping the article and removing it from the object. In some embodiments, the system further includes an energy emitting apparatus for applying the energy.

### BRIEF DESCRIPTION OF THE DRAWINGS

The principles and operations of the systems, apparatuses and methods according to some embodiments of the present disclosure may be better understood with reference to the drawings, and the following description. The drawings are given for illustrative purposes only and are not meant to be limiting.

FIGS. 1A and 1B are a schematic illustration of an exemplary detachable adhesive composition at a first inactive mode (FIG. 1A) and at a second active mode (1B), constructed and operative according to some embodiments of the present disclosure;

FIGS. 1C and 1D are a schematic illustration of an exemplary detachable adhesive composition at a first inactive mode (FIG. 1C) and at a second active mode (1D), constructed and operative according to some embodiments of the present disclosure;

FIG. 2 is an illustration of an exemplary flow chart of the processing and operation of a detachable adhesive composition, constructed and operative according to some embodiments of the present disclosure;

FIG. 3, is detachable adhesive system, constructed and operative according to some embodiments of the present disclosure; and

FIGS. 4-13 are images showing exemplary detachable adhesive compositions, constructed and operative according to some embodiments of the present disclosure.

### DETAILED DESCRIPTION OF SOME OF THE EMBODIMENTS

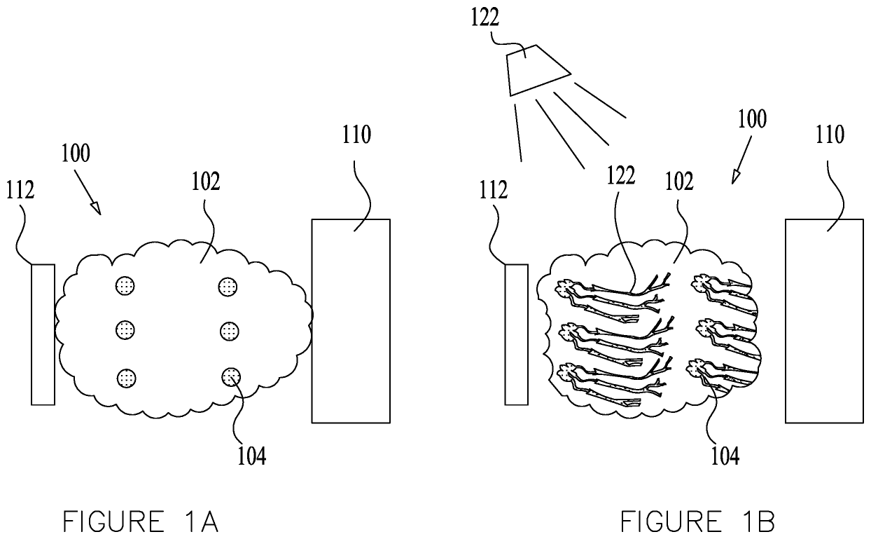

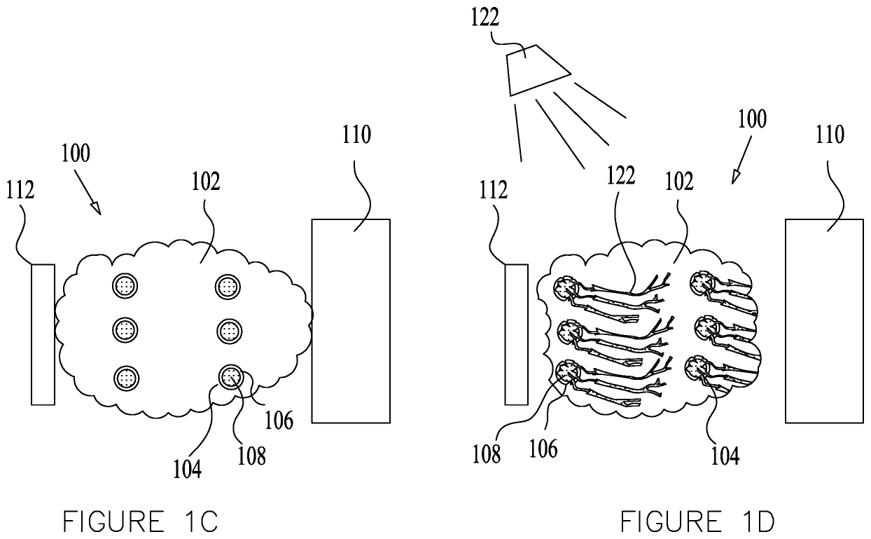

FIGS. 1A and 1B are schematic illustrations of an exemplary detachable adhesive composition 100 at a first inactive mode (FIG. 1A) and at a second active mode (1B), not shown to scale. The detachable adhesive composition 100 comprises an adhesive compound 102 and at least one or more selectively activated particles 104 (referred to herein as “particles”) dispersed within the matrix of the adhesive compound 102. The adhesive compound 102 comprises any binding material operable to adhere to an object 110. Adherence to the object 110 may be performed to bind an article 112 to the object 110, or a first object to other objects 110 or to fill cavities or gaps with the object 110 (or article 112).

As shown in FIG. 1A, during an inactive mode of the particles 104 within the detachable adhesive composition 100, the detachable adhesive composition 100 may be used to adhere to the object 112. As shown in FIG. 1B, energy is generated by a source of energy 120 and is absorbed by, or in any other way affects directly or indirectly, the particles 104, thereby transitioning the particles 104 from the first, inactive mode to a second, active mode. The source of energy 120 may comprise an energy emitting apparatus. The absorption of energy causes a physical reaction by expansion of the particles 104, thereby applying pressure and creating a cascade of fractions, fissures, or micro-cracks 122 in the adhesive compound matrix. The particles 104 may release a fluid or transform into a fluid (i.e a liquid or gas), following the energy absorption. The fluid flows into the micro-cracks 122 forming interface regions of the fluidic-selectively activated particles 104 with the adhesive compound matrix. The particles 104 comprise an active element which induces an additional reaction (besides the physical reaction) with the adhesive compound matrix. The additional reaction is determined by the properties of the active element and/or that of the adhesive compound 102.

It is noted that for simplifying the description and without limiting the scope, the energy is described as being absorbed by the particles 104, yet it is to be understood that energy absorption is intended to include embodiments wherein the energy generally affects the particles 104 directly or indirectly (e.g. through a medium).

In some embodiments, the active element may comprise a reagent and the induced additional reaction is a chemical reaction with the adhesive compound 102, acting as a reactant. The chemical reaction results in the digestion of the adhesive compound matrix at the micro-cracks 122 causing the detachable adhesive composition 100 to at least partially disintegrate or weaken. This prompts the detachment of the detachable adhesive composition 100 from the object 110. In some embodiments, the active element may comprise an oleaginous substance and the additional reaction comprises lubrication of the adhesive compound matrix at the micro-cracks 122, which weakens the adhesive compound matrix, prompting the detachment of the detachable adhesive composition 100 from the object 110.

Accordingly, the article 112 or second object or filling may be removed from the object 110. In some embodiments, the detachment of the detachable adhesive composition 100 from the object 110 is performed without any residual material remaining on the object 110 and/or article 112. In some embodiments, some residue may remain yet the article 112 is removable from the object 110.

In some embodiments, the interaction of the particles 104 with the adhesive compound matrix prompts the chemical reaction in addition to lubrication, while in some embodiments one of the chemical reaction or lubrication occurs.

In some embodiments, any one of the chemical reaction or lubrication may occur exclusively without the physical reaction.

The extensive cascade of micro-cracks 122 within the adhesive compound matrix provides for many interface regions with the particles 104 and thereby accelerates the induction of the additional reactions.

The adhesive compound 102 may comprise one or more substances including a glue, cement, concrete, binder, bonder, mucilage, paste, varnish, enamel, lacquer, polish, a hardening substance and a resin. The adhesive compound 102 may include one or more of a polymer substance, ceramic substance, metallic substance or composites.

Further non-limiting examples of the adhesive compound 102 may include cement mixtures, cement compounds, glue compounds, glue mixtures, bond compounds, bond mixtures, resin compounds, resin mixtures, epoxies, cyanoacrylates, spray adhesives, pressure sensitive adhesives, fabric adhesives, hot glue, yellow wood glue, polyurethane adhesive, synthetic polymer-comprising adhesives, synthetic monomer-comprising adhesives, solvent-comprising adhesives, plant based adhesives, animal based adhesives, mineral based adhesives, Portland Cement, sealers and bioceremic (BC) sealers.

In some embodiments, the adhesive compound 102 may comprise an adhesive used to coat or cover an object 110, such as paint or plaster, for example.

In some embodiments, the adhesive compound 102 may include any material comprising an adhesive, such as pressure sensitive tapes (PSA tapes), tapes, adhesive tape, self-stick tape, sticky tape, duct tape or wallpaper.

In some embodiment, the adhesive compound 102 may be composed by combining an adhesive and a hardening substance or any other plurality of substances or admixtures, which upon combination thereof mutually react to form the adhesive compound 102 with adhesive functionality. The substances may be combined prior to use thereof (i.e prior to attachment to the object 110) and before or after introduction of the particles 104.

In some embodiments, the adhesive compound 102 is configured to acquire its adhesiveness, namely its adhesive functionality for enabling it to adhere to the object 110, by combining two or more substance of the adhesive compound 102 and/or following a treatment. In other words, the adhesive compound 102 or one of its substances is first configured without adhesiveness and after combination with another substance and/or a treatment, it acquires its adhesiveness. This configuration is to prevent the adhesive compound 102 from adhering prematurely to an undesired object or to prematurely harden or dry. The treatment may include at least one of: exposure to light (e.g. light curing), UV (ultraviolet) curing, LED (Light-Emitting Diode) curing, application of pressure, heating, exposure to air or any other suitable treatment.

In some embodiments, a first and second (or more) substance may be contained in separate first and second (or more) substance chambers within a container containing the adhesive compound 102. For example, a first chamber may comprise an epoxy which is mixed with a hardening agent placed in a second chamber. The first and second chamber may be positioned in a single container. In some embodiments, the substances may be first placed in separate containers or applicators and mixed prior to use. In some embodiments first and second (or more) solid flexible substances may be one on top of the other , in layers or otherwise one at the center of the other and can be mixed together like a dough mixture.

In some embodiments, the particles 104 are configured to be generally inert and not react with the adhesive compound 102, or any one of the adhesive compound 102 substances and the surroundings, before the particle 104 activation (i.e. by absorption of energy). The inert particles 104 do not interfere with the activity of the adhesive compound 102 and allow the detachable adhesive composition 100 to maintain its adhesive functionality and thus be attachable to the object 110.

The surroundings may include any one or more of the materials forming the particles 104, the adhesive compound 102, the object 110 or the article 112 and/or an environment including any one of the detachable adhesive composition 100, object 110 and/or article 112. For example, when the detachable adhesive composition 100 is used in dentistry the surroundings may include particles 104, the adhesive compound 102, the dental articles, the dental object and the environment, i.e. the mouth. The particles 104 are configured to be at least partially inert in respect to these surroundings during the inactive mode.

It is noted that the detachable adhesive composition 100 includes the combination of the particles 104 with the adhesive compound 102 or any one of the adhesive compound substances, before the treatment, after the treatment or without treatment. The detachable adhesive composition 100 may include the combination of the adhesive compound 102 or any one of the adhesive compound substances with the particles 104 in the particle inactive mode or active mode. FIGS. 1A and 1C show the detachable adhesive composition 100 with its adhesive functionality, yet the detachable adhesive composition 100 also may include the combination of the particles 104 with the adhesive compound 102 before acquiring its adhesive functionality (e.g. before being subjected to a treatment or before introducing a hardening substance).

In some embodiments, the adhesive compound 102 may be formed in a preprocessing stage wherein one or more substances of the adhesive compound 102 may be treated and/or may be combined with another substance.

The detachable adhesive composition 100 is formed in a processing stage by combining the particles 104 with the adhesive compound 102, which may or may not have been preprocessed. The detachable adhesive composition 100 may also be formed in the processing stage by combining the particles 104 with one or a portion of the substances of the adhesive compound 102. Further substances may be added and a treatment may or may not be performed.

The combination of the preprocessed substances or one substance or some or all substances with the particles 104 at the processing stage is performed at a processing rate. In such a combination of substances, with or without the treatment, the particles 104 may be slightly activated yet at least partially inactive, such that their reaction rate with the adhesive compound 102 (or its substance) is slower than the processing rate of the combination of substances. This ensures that the particles 104 will not weaken the detachable adhesive composition 100 during their inactive mode, while the detachable adhesive composition 100 should maintain its adhesive functionality.

The particles 104 may be selectively flowable and may be further configured to be activated upon absorption of the energy. The energy absorption causes at least a portion of the particles 104 to flow and react with the adhesive compound 102, by the physical reaction and by any one or more of the additional reactions (e.g. a chemical reaction and/or lubrication). These reactions weaken the adhesive compound matrix and eventually prompt the detachment of the detachable adhesive composition 100 from the object 110.

According to some embodiments, the particles 104 may comprise a substance configured during the inactive mode to be, at least partially, in a solid state or confined by a solid state barrier (e.g. a microencapsulated shell) and transition to an at least partial fluid (e.g. liquid and/or gaseous) state or flow out of the barrier, in the active mode by absorption of energy. In some embodiments, the adhesive compound 102 may be configured to remain substantially in its solid state during both the inactive mode and the active mode until the adhesive compound 102 weakens or discomposes. In some embodiments, the adhesive compound 102 may be configured to be at least partially fluid during the active mode.

The reaction of the adhesive compound 102 (denoted by “A”) with the particles 104 (denoted by “B”) during transition from the inactive mode to the active mode may be described as follows in Formula 1:

A(s)+B(s)+(ACTIVATION of B)→A′(s)+B′(l/g) Formula 1

Where: “s” represents the solid state ;“l” represents the liquid state and “g” represents the gaseous state. “l/g” represents a state which may be liquid and/or gaseous or a partial liquid and partial gaseous state.
ACTIVATION of B is initiated by absorption of the energy by the particles 104.
“A′ ” and “B′ ” represent the adhesive compound 102 and the particles 104, respectively, at the active mode, upon weakening and/or decomposition of the detachable adhesive composition 100.

In some embodiments, the detachable adhesive composition 100 comprises the combination of the adhesive compound 102 and the particles 104 in the following stages (for example): the processing stage, as well as through the inactive stage, where the adhesive compound 102 is combined with the particles 104 as expressed by: A(s)+B(s) in Formula 1; and the transitioning stage, where the particles 104 transition from the inactive mode to the active mode, as expressed by: A(s)+B(s)+(ACTIVATION of B); and during the active mode, as expressed by: A′(s)+B(l/g).

While Formula 1 is a general description of the detachable adhesive composition 100, according to some embodiments, the combination of the adhesive compound 102 and the particles 104 may be performed in several stages, as described in reference to Formulae 2-7.

As described, at times the adhesive compound 102 is formed in a pre-processing stage (i.e., prior to the introduction of particles 104) by combining one or a plurality of substances, a₁+a₂ . . . aₙ, namely aᵢ, as described by Formula 2. In some embodiments, the one or more of the substances, aᵢ, may be also subjected to a treatment to produce the adhesive compound 102, as described by Formula 3. The resultant preprocessed adhesive compound 102 is denoted by A*,

a_{1(s/l/g)}+a_{2(s/l/g)} . . . a_{n(s/l/g)}→A*(s)+O(g) Formula 2

The substances aᵢ and aₙ may be in a solid, liquid or gaseous state.
The combination or treatment of the substances may produce a byproduct “O(g)”, such as a gas, which is generally diffused away from the adhesive compound 102.
In some embodiments, the byproduct “O” is not produced.
At times the substances aᵢ are treated by a treatment to produce the adhesive compound 102, A*.

The reaction due to the combination and treatment of the substances aᵢ for producing the adhesive compound 102, A*, is described in Formula 3:

a1_{(s/l/g)}+a2_{(s/l/g)}+(TREATMENT of aᵢ)→A*(s)+O(g) Formula 3

The substances aᵢ may be in a solid, liquid or gaseous state.

In some embodiments, a single substance or a plurality or all of the substances aᵢ may be combined together at a processing stage with the particles 104 (described by Formula 4):

a_{1(s/l/g)}+a_{2(s/l/g)}+B(s))→A*(s)+B(s)+O(g) Formula 4

In some embodiments, a single substance or a plurality of the substances aᵢ may be first combined at a processing stage with the particles 104, expressed by (a_{1(s/l/g)}+B(s)), and thereafter further combined with another substance, expressed by a_{2(s/l/g)} (e.g. a hardening substance, generally shortly prior to use) (described by Formula 5):

(a_{1(s/l/g)}+B(s))+a_{2(s/l/g)}→A*(s)+B(s)+O(g) Formula 5

In some embodiments, during the processing stage as described by Formula 4 the single substance or plurality or all of the substances aᵢ and/or the particles 104 may also be subjected to a treatment (described by Formula 6):

a_{1(s/l/g)}+a_{2(s/l/g)}+B(s)+(TREATMENT of aᵢ)→A*(s)+B(s)+O(g) Formula 6

In some embodiments, during the processing stage as described by Formula 5 the single substance or plurality or all of the substances aᵢ mixed with the particles 104 may be also be subjected to a treatment (described by Formula 7):

(a_{1(s/l/g)}+B(s))+a_{2(s/l/g)}+(TREATMENT of (a_{1(s/l/g)}))→A*(s)+B(s)+O(g) Formula 7

During these processing stages described by Formulae 4-7, the particles 104, B(s), may be at least partially inert with respect to the single or plurality of substances and their surroundings and other products of the processing stage like O(g), and all during the inactive mode. The inert particles 104 are denoted by the non-asterisked, solid state “B(s)”.

For transitioning from the inactive mode to the active mode, the particles 104 are activated by, for example, exposure to energy (denoted by “ACTIVATION of B”), for transitioning the particles 104 from a solid state to a fluid state or for rendering the particles 104 flowable. This is denoted by “l/g,” showing the particles 104 may be in a liquid or gaseous state or a combination thereof.

The reaction of the preprocessed or processed adhesive compound 102 (denoted by “A*”) with the particles 104 (denoted by “B”) may be described by Formula 8 (equivalent to Formula 1):

A*(s)+B(s)+(ACTIVATION of B)→A′(s)+B′(l/g) Formula 8

A*(s)+B(s) represent the detachable adhesive composition 100 in its inactive mode and A′(s)+B′(l/g) represent the detachable adhesive composition 100 in its active mode.

In some embodiments, the byproduct “O” may participate in any one of the reactions (e.g. physical, chemical and/or lubrication). For example, wherein “O” is a gas, its diffusion may cause the expansion of the particles 104 and or may cause pressure to be applied to the adhesive compound 102, thereby forming the micro-cracks 122, and further reacting as described herein.

According to some embodiments, the detachable adhesive composition 100 may be used in construction and may be formed of the adhesive compound 102 comprising a construction cement and the particles 104.

A simplified exemplary construction cement comprises concrete at a solid state, such as including at least calcium hydroxide Ca(OH)₂(s). The concrete may be formed at a preprocessing stage by adding water H₂O(l) to solid Portland Cement, here represented for simplicity by calcium oxide in its solid state, CaO(s), and can be described in Formula 9 (equivalent to general Formula 2, yet here shown without the byproduct “O”):

CaO(s)+H₂O (l)→Ca(OH)₂(s) Formula 9

In some embodiments, the concrete may be combined with the particles 104 at a processing stage to form the detachable adhesive composition 100.

In some embodiments, the Portland Cement is first combined with the particles 104, prior to introduction of the water so as to prevent premature hardening of the concrete. In this example the particles 104 comprise a PCM (Phase change Material) which are combined with the Portland Cement while the PCM is in its solid state, as described in Formula 10:

CaO(s)+PCM(s)→(CaO(s)+PCM(s)) Formula 10

At this stage the PCM remains inert and in a solid state and does not interact with the particles of the Portland Cement. Thereafter, the Portland Cement and PCM mixture (CaO(s)+PCM(s)) may be combined with water to form the concrete with its adhesive functionality. The concrete is represented here by calcium hydroxide Ca(OH)₂ for simplicity. Typically, the PCM particles are embedded in the concrete matrix. The reaction is described as follows in Formula 11 (equivalent to general Formula 5):

(CaO(s)+PCM(s))+H₂O (l)→Ca(OH)₂(s)+PCM(s). Formula 11

In both stages described by Formulae 10 and 11, the PCM remains in a solid state and does not interact with the Portland Cement matrix or with the solid concrete.

To transition from the inactive mode to the active mode, energy is applied to the concrete and absorbed by the PCM particles. The reaction of the concrete with the activated PCM during this transition from the inactive mode to the active mode may be described as follows by Formula 12 (equivalent to general Formula 8):

Ca(OH)₂(s)+PCM(s)+(ACTIVATION of PCM)→Ca(OH)₂(s)+PCM(l) Formula 12

The PCM transitions from a solid state to a liquid state and forms micro-cracks 122 in the concrete. The PCM flows into the micro-cracks 122 thereby reacting with the concrete and eventually inducing the concrete disintegration. In this simplified, non-limiting example the chemical reaction is a Base-Acid reaction.

The activation of the particles 104 is performed by exposure to energy. The energy source 120 may be configured to generate the energy at or above predetermined parameters for transitioning the particles 104 from the inactive mode to the active mode and further to ensure the transition is effected only when required (i.e. when the detachable adhesive composition 100 is to be detached) and not prematurely (i.e. when the detachable adhesive composition 100 is to remain attached to the object 110 or before attachment).

These predetermined parameters may comprise, but are not limited to, any one or more of: a predetermined energy intensity level configured to activate the particles 104; a predetermined temperature configured to activate the particles 104; a predetermined duration of exposure configured to activate the particles 104; a predetermined ultrasonic frequency configured to activate the particles 104; a predetermined electromagnetic frequency configured to activate the particles 104. The electromagnetic frequency may be configured to resonate with the particles 104 at a predetermined frequency, such as a predetermined wavelength, e.g. a microwave frequency or an optical wavelength frequency.

In some embodiments, to prevent the particles 104 from prematurely reacting with the adhesive compound matrix during the inactive mode and/or to prevent the particles 104 from unintended altering of the properties of the adhesive compound 102, the total weight or volume of the particles 104 within the detachable adhesive composition 100 may be preferably small relative to the weight or volume of the adhesive. In a non-limiting example, the weight or volume of the detachable adhesive composition 100 is formed of about 1% to 40% particles 104 and, correspondingly, about 99%-60% of the adhesive compound 102, as well as variables and subranges thereof. In a non-limiting example, the weight or volume of the detachable adhesive composition 100 is formed of about 1% to 10% particles 104 and, correspondingly, about 99%-90% of the adhesive compound 102, as well as variables and subranges thereof. In a non-limiting example, the weight or volume of the detachable adhesive composition 100 comprises about 5% particles 104 and, correspondingly, about 95% of the adhesive compound 102. In a non-limiting example, the weight or volume of the detachable adhesive composition 100 comprises about 3% particles 104 and, about 97% of the adhesive compound 102.

In a non-limiting example, the weight or volume of the detachable adhesive composition 100 is formed of about 1% to 80% adhesive compound 102 and, correspondingly, about 99%-20% of the particles 104, as well as variables and subranges thereof.

According to some embodiments, the detachable adhesive compound 100 may comprise a relatively thin layer for sufficiently attaching to an object 110 or adhere an article 112 to an object 110. For example, the detachable adhesive compound 100 may comprise a layer of about a few millimeters to a single centimeter to a few centimeters. In some examples, the thickness of a layer of standard concrete is 10 centimeters.

The reaction rate of the additional reactions, e.g., the chemical reactions and/or lubrication is affected by a level of a reacting parameter (RP) of the adhesive compound 102 and the RP level of the particles 104. Such an RP may be, for example, a pH level of the adhesive compound 102 and the particles 104.

In some embodiments, during the inactive mode of the particles 104, the difference (denoted by ΔRP) between the RP level of the adhesive compound 102 (or at least one of its substances aᵢ) and the RP level of the particles 104 is relatively small, such that the reaction rate of the particles 104 with the adhesive compound matrix is slow or zero. During the active mode the ΔRP is relatively large, such that the reaction rate of the particles 104 with the adhesive compound matrix is accelerated. The RP level may be measured in RP units.

In a non-limiting example, where the RP level is the pH level, the adhesive compound 102 is basic, i.e., pH>7, in its natural/original state (i.e. prior to undergoing any one of the reactions) and the particles 104 are acidic, i.e., a pH<7, in their natural/original state. The chemical reaction comprises a Base-Acid reaction. During the inactive mode the difference between the pH level of the adhesive compound 102 and the pH level of the particles 104 (denoted by ΔpH) is relatively small, such as in the range between 1-4 ΔpH. During the active mode the ΔRP is relatively large, such as in the range of 5-11 ΔpH.

In a non-limiting example, the chemical reaction comprises a Base-Acid reaction. The adhesive compound 102 may comprise a base and the particles 104 may be acidic, for example. In some embodiments, the particles 104 may comprise a base and the adhesive compound 102 may be acidic, for example.

In a non-limiting example, the chemical reaction comprises a Salt-Acid reaction. The adhesive compound 102 may comprise a salt and the particles 104 may be acidic. In some embodiments, the particles 104 may comprise a salt and the adhesive compound 102 may be acidic, for example.

In a non-limiting example, the chemical reaction comprises an Alkaline-Acid reaction. The adhesive compound 102 may comprise an alkaline and the particles 104 may be acidic. In some embodiments, the particles 104 may comprise an alkaline and the adhesive compound 102 may be acidic, for example.

In a non-limiting example, the chemical reaction comprises ion exchange. The ARP may comprise an ion exchange capacity which measures the ability of the particles 104 to undergo displacement of ions previously attached and incorporated into its structure by oppositely charged ions present in the adhesive compound matrix. The ion exchange capacity may be expressed in units of eqv/L (equivalents per Liter of substance) in an non-limiting example. During the inactive mode the ion exchange capacity is relatively small. During the active mode the ion exchange capacity is relatively large.

In some embodiments, the particles 104 are uniformly dispersed within the detachable adhesive composition 100. For a non-limiting example, uniformity may be achieved by mixing the particles 104 with particulates of the adhesive compound 102 to a degree such that the ratio of weight or volume of the particles 104 to that weigh or volume of the particulates forming the adhesive compound 102 is generally the same throughout the volume of the detachable adhesive compound 100.

Uniform dispersion of the particles 104 within the detachable adhesive composition 100 increases the interaction regions within the micro-cracks 122 and accelerates the reaction rate during the active mode. Furthermore, uniformly dispersed interaction regions increase the decomposition of the adhesive compound matrix substantially throughout its entire volume. Accordingly, the detachment of the detachable adhesive composition 100 from the object 110 can be deployed with little or no residual remains.

The particles 104 may be formed in the inactive mode as a powder or any other granular substance. As the granules are smaller, greater uniformity of dispersion is enabled. The granule size may be determined by the characteristics of the object 110 and article 112. In a non-limiting example, the particles 104 comprise granules of a diameter of 300 microns or less. In a non-limiting example, the particles 104 comprise granules of a diameter of 100 microns or less. In a non-limiting example the particles 104 comprise granules of a diameter of 50 microns or less. In a non-limiting example the particles 104 comprise granules of a diameter of 5 microns or less.

In some embodiments, the particles 104 are un-encapsulated. During the inactive mode the particles 104 are disposed in a solid state within the adhesive compound matrix in direct contact therewith. Following the absorption of energy, the particles 104 transition into the liquid and/or gaseous state and flow into the micro-cracks. The unencapsulated particles 104 are shown in FIGS. 1A and 1B.

According to some embodiments, the particles 104 comprise a microcapsule formed of a microcapsule shell 106 housing an encapsulated material 108, as shown in FIGS. 1C and 1D.

In some embodiments the microcapsule shell 106 is formed of a solid material which is configured to remain solid during the inactive mode and rupture or dissolve during the active mode. An exemplary solid material may comprise silica or plastic. The encapsulated material 108 may comprise a PCM or any other material configured to be in a solid or liquid state during the inactive mode.

In some embodiments the encapsulated material 108 is a solid in its inactive state. Following the absorption of energy, the encapsulated material 108 transitions into the liquid and/or gaseous state. The now fluid encapsulated material 108 expands and presses upon the surface of the microcapsule shell 106, which in turn presses upon the adhesive compound matrix forming the micro-cracks 122. Furthermore, the microcapsule surface (namely its shell) 106 may rupture allowing the encapsulated material 108 to egress the microcapsule shell 106 and flow into the micro-cracks 122.

In some embodiments, the encapsulated material 108 may be in a liquid state (e.g. water) or gaseous state during the inactive mode. Following the absorption of energy, fluid encapsulated material expands and presses upon the surface of the microcapsule shell 106, which in turn presses upon the adhesive compound matrix forming the micro-cracks 122.

In some embodiments, the activation may itself also rupture or dissolve the shell 106 allowing the encapsulated material 108 to egress the microcapsule 106 and flow into the micro-cracks 122.

In some embodiments, the microcapsule shell 106 is formed of a PCM which is transitioned, at least partially, from a solid during the inactive mode to a liquid or gas during the active mode. The encapsulated material 108 may comprise a PCM or a non-PCM material, which maintains its solid or liquid or gaseous state during the inactive and active modes.

In some embodiments, the microcapsule shell 106 is formed of a non-PCM material which is transitioned, at least partially, from a solid during the inactive mode to a liquid or gas during the active mode. For example, the shell 106 may comprise a plastic configured to absorbed energy generated at a predetermined wavelength, causing the shell 106 to melt or rupture.

In some embodiments, the encapsulated material 108 may comprise a fluid, such as water or a viscous fluid or gel. The fluid may be at its liquid phase (at least partially) during the inactive mode and may vaporize to a gaseous phase (at least partially) or evaporate upon absorption of energy in the active mode.

In some embodiments, the particles 104 may comprise a PCM, in any configuration such as un-encapsulated or in the encapsulated material 108 and/or in the microcapsule shell 106 (such a PCM shell may comprise paraffin for example). The PCM is configured to be initially at a first state (i.e. a phase) and following adsorption of the energy, transition into another state at or above a transitional temperature (namely, the phase change temperature). In some embodiments, the PCM phase change can be reversed upon emission of energy from the PCM. The PCM may be configured as latent heat storage units with a high heat of fusion, capable of storing and releasing large amounts of energy (generally as heat).

The PCM may include any one of an inorganic PCM comprising, for example, at least one of salt hydrates, salts, metals and alloys; or an organic PCM comprising, for example, at least one of a paraffin, fatty acids, oils, biocompatible oils, vegetable oils, alcohols and glycols and an oleaginous substance. In some embodiments, the PCM may comprise free fatty acids, such as palm oil, palm kernel oil, rapeseed oil, coconut oil and soybean oil, for example.

In some embodiments, the particles 104 may be formed of a single or plurality of materials. For example, an un-encapsulated particle 104 or the encapsulated material 108 may comprise two or more materials, all solid, all fluid or some solid and some fluid. In some embodiments, these plurality of materials, comprising a first or second or more material, may be configured to be at least partially inert to their surroundings at any stage prior to their activation at the activation mode. The activation may cause a first material to internally react (physically, chemically and/or by lubrication or any other reaction) with a second material. This internal reaction may thereafter induce the expansion of the particles 102 causing the formation of micro-cracks 122.

In some embodiments, the particles 104 may be configured to transition from the inactive mode to the active mode at a predetermined transitioning temperature. The energy may be generated from the energy source at or above that predetermined transitioning temperature. In a non limiting example, the predetermined transitioning temperature may span from −40 to+150° C., any subranges or variants thereof.

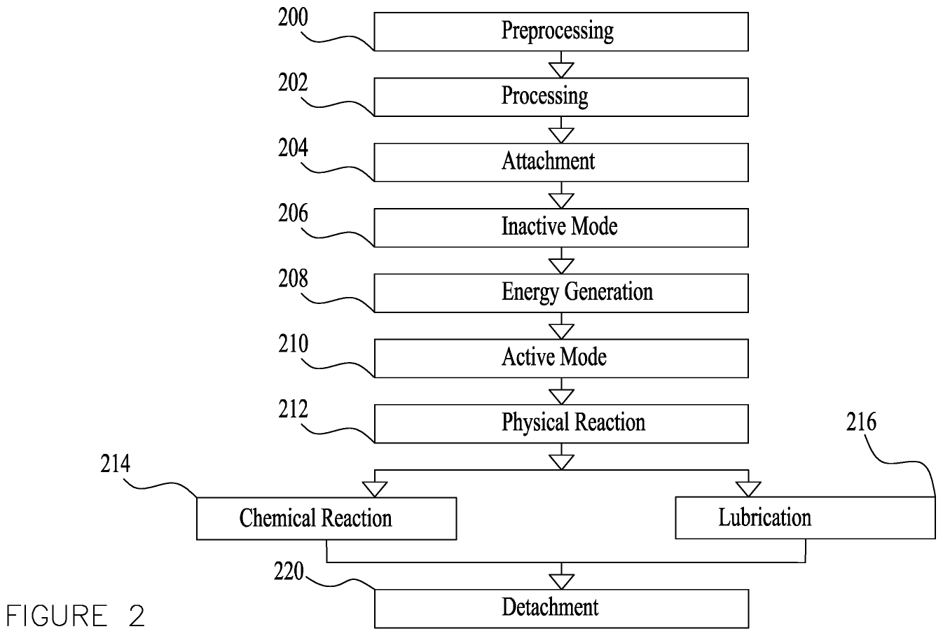

FIG. 2 is an illustration of an exemplary flow chart of the processing and operation of a detachable adhesive composition, according to some embodiments. As seen at step 200, the adhesive compound 102 may be first formulated at a preprocessing stage by combining two or more substances, prior to introducing the particles 104. The two or more substances may be kept separate and are only mixed close to their use as adhesives so as to prevent the adhesive compound 102 from drying prior to use on the object 110. In some embodiments, the adhesive compound 102, comprising a single substance or a plurality of substances, may be subjected to a treatment. As described herein, the treatment may include at least one of: exposure to light (e.g. light curing), UV curing, LED curing heating, exposure to air, applying pressure or any other suitable treatment.

In some embodiments:
the combination of the substances and optional treatment may be performed a long or short time before use of the adhesive compound 102;
the preprocessing stage 200 may comprise premixing the plurality of substances and providing the resultant mixture in an applicator or any other container;
the preprocessing stage 200 may be omitted and the adhesive compound 102 may be provided in its final composition; and/or
the preprocessing stage 200 may be omitted and substances of the adhesive compound 102 may be combined with the particles 104 at a plurality of stages during a processing stage.

At processing step 202, the particles 104 may be introduced into the adhesive compound 102 in any suitable manner, such as by mixing, embedding, impregnating or pressing for producing the detachable adhesive composition 100.

In some embodiments, the adhesive component 102 comprises the plurality of substances, a₁+a₂ . . . aₙ, which are kept separate and are only mixed close to their use as , so as to prevent the mixture from drying or hardening prior to use on the object 110. The plurality of substances may be mixed with the particles 104 at the processing step 202 at several stages. Initially the particles 104 can be combined with a more inert substance and thereafter be mixed with a more active substance, to maintain the inert state of the particles 104 during the inactive mode. For example, an adhesive component 102 is formed by combining a first substance with a pH of 4 and a second substance with a pH of 7. In some embodiments, for particles 104 with a pH of 9, it may be best to select the more neutral substance, i.e. the second substance, to be first mixed with the particles 104. At a later stage prior to use (to attach to the object 110) the mixture of the second substance with the particles 104 is mixed with the first substance.

At step 204, the detachable adhesive composition 100 may be attached to the object 110 and/or article 102. The particles 104 are configured, through the duration from preprocessing step 200, processing step 202, and the inactive mode step 206, to remain nonreactive with the adhesive compound 102, so as to ensure the detachable adhesive composition 100 is securely attached to the object 110.

When the removal and detachment of the detachable adhesive composition 100 from the object 110 is desired, energy is generated/supplied from an energy source 120, as shown at step 208. The energy may comprise at least one of thermal energy, sonic energy, ultrasonic energy, electromagnetic energy, UV, light, microwave, and any type of radiation that interacts with the particles 104 causing them to transition to the active mode or any type of radiation causing the particles 104 to resonate at a predetermined frequency. For example, the energy source 120 may comprise at least one of a heat-operated device, a sonically-operated device, an electromagnetically-operated device, a UV-operated device (e.g. a UV light lamp), an optically-operated device, a microwave-operated device, and a radiation-operated device for example. In some embodiments, the energy may be configured to generate non-mechanical energy.

The energy may be generated at an intensity and/or a duration required for transitioning the particles 104 from the inactive mode to an active mode. In a non-limiting example, the energy may be supplied/emitted for a duration of 60 seconds, less or more, or 120 seconds or less or more, or 5 minutes or 10 minutes, or a few second, 5 second, 15 seconds or a few minutes to an hour or hours, more or less and subranges and variants thereof. In some embodiments, more than one source of energy may be used of the same type of energy (e.g. two or more UV light lamp) or a combination of energy sources may be used (e.g. a heater and a UV light lamp).

The particles 104 absorb the energy and transition into an active mode, at step 210. Particles 104 comprising an un-encapsulated solid in the inactive mode transition into a fluid during the active mode. The particles 104 may comprise the microcapsule formed of the shell 106 housing the encapsulated solid or fluid material 108 in the inactive mode. The absorption of energy may cause the shell 106 to rupture and release the encapsulated fluid material 108 and/or transition the encapsulated solid material 108 into a fluid, during the active mode.

Moreover, during the active mode, the absorption of energy causes a physical reaction by expansion of the particles 104, thereby applying pressure and creating the cascade of micro-cracks 122 in the adhesive compound matrix, as shown at step 212. The fluid (from the un-encapsulated or encapsulated material) flows into the micro-cracks 122 at the interface regions. The particles 104 react with the adhesive compound matrix prompting an additional reaction.

As seen at step 214, in some embodiments, the additional reaction is a chemical reaction which may result in the digestion of the adhesive compound matrix at the micro-cracks 122 or any other chemical reaction. As seen at step 216, in some embodiments, the additional reaction comprises lubrication of the adhesive compound matrix at the micro-cracks 122. In some embodiments both the chemical reaction and the lubrication steps may be deployed.

In a non-limiting example, the particles 104 comprise a PCM including free fatty acids, such a vegetable oil, and the adhesive compound 102 comprises a cement. The base-acid chemical reaction is deployed, thereby digesting the cement. Concurrently the fatty acids lubricate the micro-cracks 122.

The additional reactions of steps 214 and 216, deployed separately or together, are configured to weaken the adhesive compound matrix, prompting the detachment of the detachable adhesive composition 100 from the object 110, as shown at step 220.

The sequence of steps described herein may be alternated. For example, in some embodiments, the particles may first be subjected to any one or both of the additional reactions of steps 214 and 216 , and thereafter be subjected to the physical reaction of step 212. In another example, the particles 104 after absorption of the energy may commence to chemically react with the adhesive compound 102 , which may induce the physical reaction, such as the expansion and the formation of micro-cracks 122. In some embodiments, any one or more of the reactions 212 (physical), 214 (chemical) or 216 (lubrication) may be eliminated .

Accordingly, in some embodiments, there is provided a method for selectively adhering the detachable adhesive composition 100 to an object 110 and detachment therefrom without requiring direct contact or application of a mechanical force to detach the detachable adhesive composition 100. In such embodiments, the detachable adhesive composition 100 can be selectively detached from an object 110 non-mechanically. Alternatively, the detachable adhesive composition 100 can be selectively detached from an object 110 by using a mechanical force, yet smaller than a mechanical force which would have been required for detachment from the object 110 in absence of the aid of the detachable adhesive composition 100.

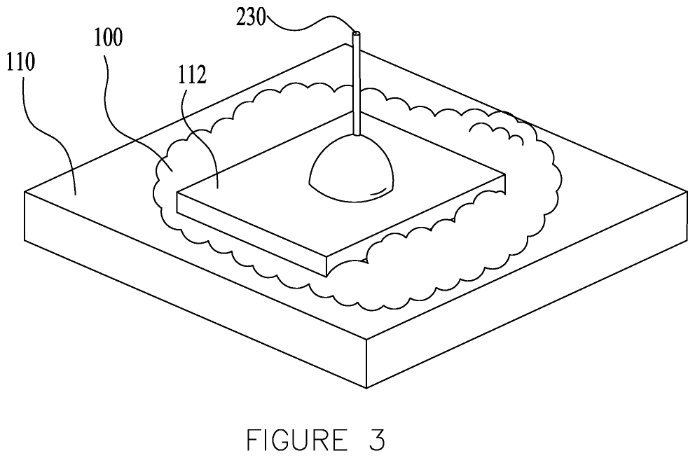

As shown in FIG. 3, there is provided a detachable adhesive system for selectively adhering the article 112 to the object 110 or filling an object 110 and detaching it therefrom. The system comprises the detachable adhesive composition 100 and may further comprise any one of the articles 112 and/or objects 110. In some embodiments, the system may comprise a griping element 230 for gripping the article 112 (or object 110), here shown as a floor tile, and removing it from the object 110 shown as the floor. The gripping element 230 may comprise suction or any other suitable features. It is noted that the gripping force required is relatively weak since the detachable adhesive composition 100 is already detached from the object 110 following generation of energy in the active mode. The force executed by the gripping force is significantly smaller (e.g. 50% or less) than the force which would have been required had only the adhesive compound 102 been used. In some embodiments, the system comprises a utility device housing both the griping element 230 and the energy emitting element 120 in the same housing. In a non-limiting example, such a utility device may include a light curing device as well as a scalpel or pincers used in dentistry.

In some embodiments, the decomposition of the detachable adhesive composition 100 may be reversed. The particles 104 may be transitioned from the active mode back to the inactive mode and recombined with the adhesive compound 102 for reformulating the detachable adhesive composition 100. The detachable adhesive composition 100 may be reused to attach (and possibly thereafter to detach) to the object 110. The particles 104 may be transitioned from the active mode back to the inactive mode in any suitable manner, such as by applying energy to reverse the particle fluid state to a solid state. In another example, exposure to low temperatures or a cool environment (e.g. refrigeration) or to the ambient environment, may cause the particles 104 to solidify. For example, whereupon the particles 104 comprise a PCM any exposure to a temperature lower than the PCM transition temperature will re-solidify the PCM. In a non-limiting example, during construction or renovation, a tile, attached to a construction surface by the detachable adhesive composition 100, may be detached while renovating and thereafter reattached for repeated use.

The detachable adhesive composition 100 may be used for a wide array of applications. Below are a few non-limiting examples.

According to some embodiments, the detachable adhesive composition 100 may be deployed in dentistry wherein the article 112 comprises at least one of a dental article including a dental crown, bracket, bridge or brace and is adhered to the object 110, which comprises any one of a dental object including a tooth, dental implant or abutment, by the detachable adhesive composition 100 which is formed with the adhesive compound 102 comprising a dental cement.

Further non-limiting examples of dental cement may comprise: BC SEALER commercially available from BRASSELER USA ®, HARVARD CEMENT commercially available from HARVARD®, RELYX, UNICEM, or DURELON commercially available from 3M, GC Fuji IX GP® commercially available from GC, or any type of Glass Ionomer Cement (GIC).

In some embodiments, the dental cement may be formed by combining a dental adhesive with a hardening substance, at a preprocessing stage. In some embodiments, the preprocessing stage may be omitted and the dental cement may be ready for use. The detachable adhesive composition 100 may be formed by combining the particles 104 with the dental cement a long time before use in a mouth or immediately prior thereto.

Initially, at the inactive mode the detachable adhesive composition 100 is used as a selectively detachable dental cement to attach the dental articles to any one of the dental objects, or the selectively detachable dental cement may be used as a filling material for filling a cavity or dental caries. The attachment may be performed solely by application of the selectively detachable dental cement, which may be configured to autonomously harden, or the attachment may be performed with the aid of a treatment, such as by light curing. In any event, during the inactive mode, as long as the selectively detachable dental cement is to remain attached, the particles 104 are configured to remain inert during the treatment or hardening. In a non-limiting example, the particles 104 may comprise a PCM with a relatively high transitional temperature, or at least higher than the temperature effected by the treatment. In a non-limiting example, the PCM transitional temperature may be in the range of about 20-55° C. In a non-limiting example, the PCM transitional temperature may be in the range of about 40-55° C. and the temperature of the particles 104 during the treatment remains under 40° C.

It may be desirable to detach dental articles or fillings from any one of the dental objects, by exposing the PCM to energy (which may be generated by UV light) to raise the temperature of the cement to its transitional temperature (e.g., in some embodiments, at 45° C.) or above. The selectively detachable dental cement is now in its active mode commencing its decomposition and eventual detachment from the dental objects. The detachment is thus painlessly performed substantially without any remaining residues.

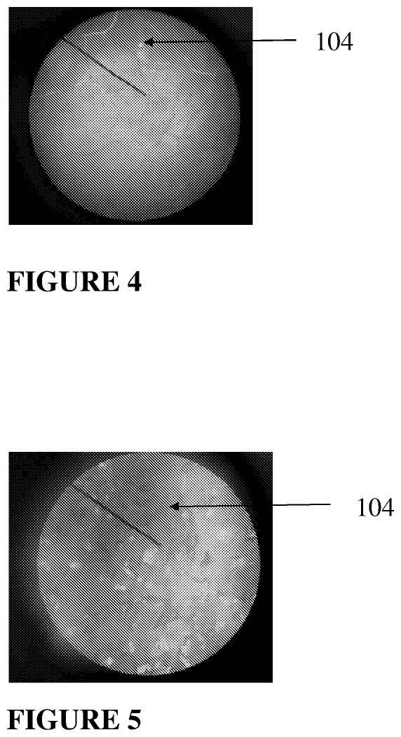

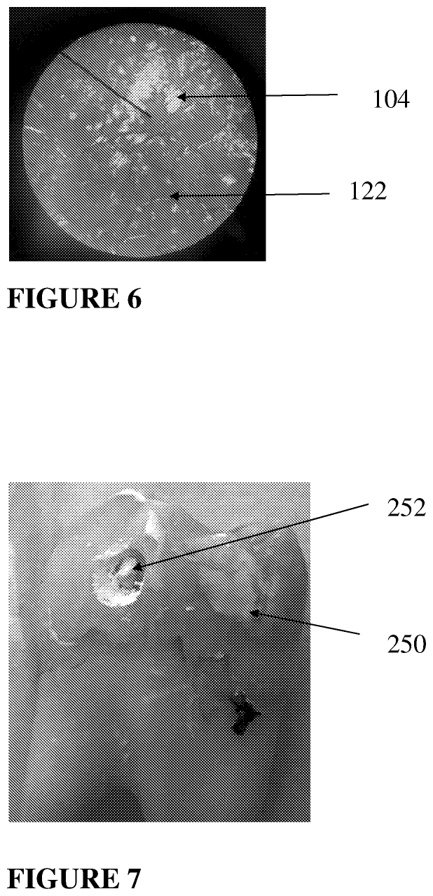

FIGS. 4-6 are micrographs imaged in a transmission optical microscope at a X100 enlargement, which show an exemplary detachable adhesive composition 100 where the adhesive compound 102 is a dental cement comprising a hard thin layer of GIC and the particles 104 are a vegetable oil-based PCM powder with a transitional temperature of 28° C. FIG. 4 shows the composition of a hard, thin layer of GIC prior to the addition of the PCM. FIG. 5 shows the hardened composition of the detachable adhesive composition 100 wherein the PCMs are mixed within the GIC matrix and appear as white particles. FIG. 5 shows the particles 104 in their inactive mode, while FIG. 6 shows the PCMs in their active mode following their exposure to infrared light emitted from an infrared lamp at a temperature above 28° C. for about 10-15 seconds. The micro-cracks 122 and flowing (i.e. melted) PCMs can be seen in FIG. 6.

FIG. 7 is an enlarged photo of a tooth with two cavities. The first right side cavity 250 was filled with conventional dental cement comprising dental HARVARD CEMENT. The second, left side cavity 252 was filled with a detachable adhesive composition 100 comprising a mixture of the dental cement and a vegetable oil-based PCM powder. The tooth was exposed to energy from an infrared lamp for about 30-75 seconds. A metal scalpel was used to remove both right and left side fillings. The left side filling 252 (filled with the detachable adhesive composition 100) is shown removed, following the discomposure of the detachable adhesive composition 100, while the right side filling 250 (filled with just the dental cement) remains in the cavity.

According to some embodiments, the detachable adhesive composition 100 may be deployed in construction wherein the article 112 comprises a tile or flooring, furniture, boards, wallpaper, as well as paints or any article of construction. The object 110 may comprise a structure, such as a floor, wall, ceiling or any structural object or surface. The adhesive compound 102 may comprise a construction cement.

A non-limiting example of a construction cement is Portland Cement, acrylic based cement, polymer or epoxy based cement materials. During reconstruction or restoration it may be required to remove the construction article while undamaging the construction article and/or structure and/or with minimal or no remaining residues.

Initially at the inactive mode the detachable adhesive composition 100 is used as a selectively detachable cement to attach the construction articles to any one of the structures, or the selectively detachable cement may be used as a filling material or for filling gaps or cavities in a structure or article.

In some embodiments, the detachable cement may be formed by combining a cement with another substance and/or water, at a preprocessing stage and/or by combining a cement with another substance and/or water and the particles 104, at a processing stage, a long time before use or immediately prior thereto.

During the inactive mode, as long as the selectively detachable cement is to remain attached, the particles 104 are configured to remain inert. In a non-limiting example, the particles 104 may comprise a PCM with a relatively high transitional temperature, or at least higher than a temperature effected by the treatment. In a non-limiting example, the PCM transitional temperature may be in the range of about 20-200° C. In a non-limiting example, the PCM transitional temperature may be in the range of about 80-200° C. In a non-limiting example, the PCM transitional temperature may be in the range of about 40-70° C.

It may be desirable to detach the construction article or filling from any one of the structures (such as during renovations), by raising the PCM temperature to its transitional temperature, via energy supplied by a heater. The selectively detachable cement is now in its active mode commencing its decomposition and eventual detachment from the structures. The detachment is thus performed substantially without any remaining residues.

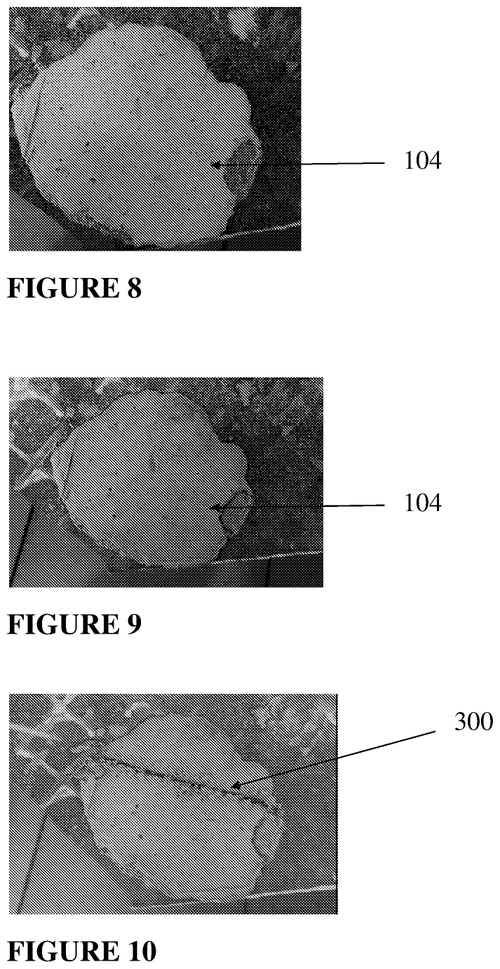

FIGS. 8-10 are micrographs imaged in a transmission optical microscope at a X100 enlargement. FIGS. 8-10 show an exemplary detachable adhesive composition 100 comprises a construction cement comprising HARVARD CEMENT for construction and particles 104. The particles 104 are a vegetable oil-based PCM powder with a transitional temperature 28° C. FIG. 8 shows the composition of the detachable adhesive composition 100 wherein the PCMs are mixed within the construction cement matrix and appear as black particles in their inactive mode. It can be observed that the material is dry, while FIG. 9 shows the PCMs in their active mode following their exposure to infrared light emitted from an infrared lamp at a temperature above 28° C. for about 90-180 seconds. It can be seen that the material is wetted by the fluid PCM and can be observed by the dark color in FIG. 9. FIG. 10 shows a scratch 300 formed in the detachable adhesive composition 100 as the detachable adhesive composition 100 decomposes.

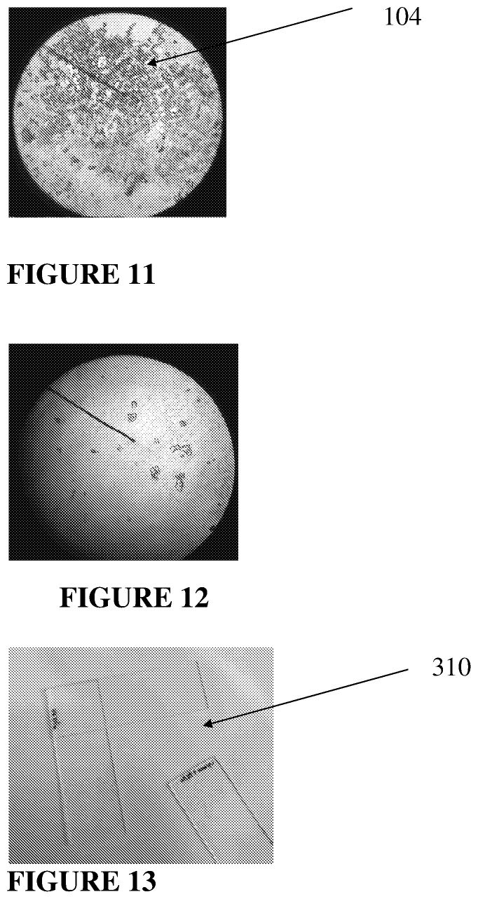

FIGS. 11-12 are transmission micrographs imaged in a transmission optical microscope showing two microscopic views at a X100 enlargement while FIG. 13 is a non-enlarged view of two glass surfaces joint together. FIGS. 11 and 12 show two glass surfaces joint together by a detachable adhesive composition 100, wherein the adhesive compound 102 comprises cyanoacrylate and the particles 104 are a vegetable oil-based PCM powder with a transitional temperature of 28° C. FIG. 11 shows the composition of the detachable adhesive composition 100 wherein the PCMs are mixed within the cyanoacrylate matrix and appear as grey particles in their inactive mode. FIG. 12 shows the PCMs in their active mode following their exposure to infrared light emitted from an infrared lamp for 30 seconds. The PCM in its liquid state is translucent and therefore invisible in FIG. 12. The micro-cracks are present yet can't be observed.

FIG. 13 is non-microscopic image illustrating the lower side glass surfaces 320 which were joined together using the detachable adhesive composition 100, following detachment. The upper side glass surfaces 310 were joined together by the cyanoacrylate only, and accordingly, even after the exposure to the infrared light, remain attached.

According to some embodiments, the detachable adhesive composition 100 may be deployed in nail treatment applications.

For example, the article 112 comprises a nail article, which may include at least one of: nail polish, gel nail polish, nail extensions, artificial nails, acrylic nails or any one of their components or layers. The object 110 comprises a human or mammal nail.

In some embodiments, nail polish comprises of any one or more of the following components or equivalents thereof: a film forming polymer dissolved in a volatile organic solvent, such as nitrocellulose. Nitrocellulose that is dissolved in butyl acetate or ethyl acetate is common. This basic formulation may be expanded to include plasticizers to yield non-brittle films. Dibutylphthalate and camphor are typical plasticizers and dyes and pigments. Nail polish may further comprise adhesive polymers to ensure that the nitrocellulose adheres to the nail's surface, such as to sylamide-formaldehyde resin.

Thickening agents may be added to the nail polish to maintain the particles in suspension while in the bottle. A typical thickener is stearalkonium hectorite. Thickening agents exhibit thixotropy, their solutions are viscous when still but free flowing when agitated. The nail polish may comprise ultraviolet stabilizers to resist color changes when the dry film is exposed to sunlight. A typical stabilizer is benzophenone.

Gel nail polish comprises any one of the components of nail polish described herein as well as methacrylate compounds and photoinitiating compounds, such as benzoyl peroxide. Photoinitiators creates reactive species, free radicals, cations or anions, when exposed to radiation (e.g. UV, LED or visible light). Gel nail polish may comprise thermochromic (temperature-sensitive) or photochromic (light-sensitive) nail polishes. Thermochromic polishes comprise leucodyes compounds contained within microcapsules. These microcapsules also contain a low melting point solvent and an acid. When the temperature is low enough, the dye and the acid molecules are in close proximity, allowing transfer of hydrogen atoms between the molecules and leaving the dye in its colored form. As temperature increases, the solvent melts, and the molecules move away from each other.

Photochromic polishes use light-sensitive compounds which experience a structural change when exposed to light. Examples of compounds used include spiropyrans and spirooxazines.

Gel nail polish may be applied in layers which are exposed to radiation to propel a polymerization process, which solidifies the polish. In some embodiments, a bonding layer is applied to the nail to adhere the remaining layers to the nails.

The nail treatment may also comprise application of artificial nails or acrylic nails, also including nail extensions, which generally are adhered to the nail by a bonding or primer layer (e.g. an acrylic primer) comprising any suitable material for adhering the nail article to the nail.

The abovementioned components and their equivalents and layers of the nail polish, gel nail polish, artificial nails, acrylic nails and nail extensions are referred to as “nail articles”.

Removal of the nail article may be difficult. For example, removal of conventional gel polish is currently performed mechanically by chipping the nail polish off the nail, generally by use of an instrument, such as a nail file. In some example, the removal of the nail article entails immersing the nail articles while on the human nail within a nail remover agent (e. g. acetone) for a long time. This process of conventional nail removal, by mechanical removal and/or by immersion in a nail remover is lengthily, arduous and may damage the nail.

In some embodiments, the detachable adhesive composition 100 may be used as a nail article or as a nail article component (e.g. in a priming/bonding layer).

Initially at the inactive mode the detachable adhesive composition 100 is used as a selectively detachable nail article or as a nail article component to attach the nail articles to the nail.

In some embodiments, the detachable adhesive composition 100 may be formed by combining a nail article component and the particles 104, at a processing stage, a long time before use or immediately prior thereto.

The nail article may be adhered to the nail by applying the nail article, or by use of a bonding/priming layer and passage of time and/or by treatment comprising exposure to radiation.

During the inactive mode, as long as the detachable adhesive composition 100 is to remain attached, the particles 104 are configured to remain inert, also during treatment. In a non-limiting example, the particles 104 may comprise a PCM with a relatively high transitional temperature, or at least higher than a temperature effected by the treatment. In a non-limiting example, the PCM transitional temperature may be in the range of about 40-60° C.

It may also be applicable to detach a nail article or nail article component from a nail, by application of energy via a heater, a UV or LED light, to raise the PCM temperature to its transitional temperature, (e.g. at 45° C. in some embodiments). The selectively detachable particles 104 are now in their active mode commencing the detachable adhesive composition 100 decomposition and eventual detachment from the nails. The detachment is thus performed painlessly, substantially without any remaining residues on the nail and without use of mechanical instruments or force and/or without use of nail remover agents.

According to some embodiments, the detachable adhesive composition 100 may be deployed in paper applications or any other application using thin materials (e.g. with a thickness of a few centimeters or less or a few microns or less). Such a paper may comprise repositionable paper typically formed with fibers of cellulose. The paper may be coated with a pressure-sensitive adhesive (PSA), such as low-tack PSA. PSAs may comprise an elastomer compounded with a suitable tackifier (e.g., a rosin ester). In some embodiments, the PSA may comprise the detachable adhesive composition 100 with or without additional components. Such paper applications may comprise wallpaper, stickers or sticky notes configured to attached to a surface. The paper may be adhered to the object 110 (e.g. a surface) by a detachable adhesive composition 100. For detachment of the paper from the surface, the detachable adhesive composition 100 may be exposed to energy, such as heat, for removal with substantially no residues from the surface.

According to some embodiments, the detachable adhesive composition 100 may be deployed in tape applications or any other application using thin materials (e.g., with a thickness of a few centimeters or less or a few microns or less). Such a tape may comprise repositionable plastic tape. The tape may be coated at one side or both sides with a pressure-sensitive adhesive (PSA), such as low-tack PSA. PSAs may comprise an elastomer compounded with a suitable tackifier (e.g., a rosin ester). In some embodiments, the PSA may comprise the detachable adhesive composition 100 with or without additional components. Such tape applications may comprise wallpaper, stickers duck tapes configured to attached to a surface. The tape may be adhered to the object 110 (e.g. a surface) by a detachable adhesive composition 100. For detachment of the tape from the surface, the detachable adhesive composition 100 may be exposed to energy, such as heat, for removal with substantially no residues from the surface.

According to some embodiments, the detachable adhesive composition 100 may be deployed in paints for adhering the paint, i.e. the article 112, to any surface, i.e. the object 110, and allowing selective removal of the paint from the surface.

At times, treatment is used to attach the article 112 to the object 110, exposing the article 112 or object 110 to a treatment temperature. It is noted that wherein the particles 104 comprise a PCM, the transitional temperature of the PCM is determined to be a temperature above the treatment temperature, to ensure the particles 104 remain inert during the treatment and while the article 112 is to remain attached to the object 110, during the inactive mode. The transitional temperature is determined to be a temperature above the treatment temperature, that will allow the detachment of the article 112 from the object 110 during the active mode.

In some embodiments, the transitional temperature may be determined to be a temperature which does not harm the vicinity (namely the environment or surroundings) of the object 110 and/or article 112. For example, in the dental application the transitional temperature of the PCM is configured to be lower than a temperature which can burn the mouth, e.g., less than 80° C. In another example, in the nail treatment application the transitional temperature of the PCM is configured to be lower than a temperature which can burn the nails or body parts, e.g., less than 60° C.

In some embodiments, the PCM type and its transitional temperature may be selected according to the anticipated deployment and application of the detachable adhesive composition 100.

In some embodiments, the energy may be generated and absorbed by the particles 104 via a medium which aids in the transition of the particles 104 from the inactive mode to the active mode. For example, wherein the detachable adhesive composition 100 is used in dentistry, the mouth may be filled with a medium, such as water or saliva. The energy from the energy source may be configured to be emitted throughout the mouth via the medium without requiring direct emission to a specific tooth or dental object. In some embodiments, the energy, such as a heat, may be delivered to the particles 104 via a medium, which heats water and the heated water, in turn, heats the particles at or above the transitional temperature.

In some embodiments, use of the detachable adhesive composition 100 allows for targeted removal and detachment of an article 112 from an object 110 by exposing only a predetermined portion of the detachable adhesive composition 100. Accordingly, only the predetermined portion is transitioned from an inactive mode to the active mode, and only in the predetermined portion is the detachable adhesive composition 100 removed from the object 110.

In some embodiments, the physical reaction described herein may comprise a mechanical reaction effecting the mechanical properties of the detachable adhesive composition 100, such as by expansion and/or application of pressure, strain or stress on the adhesive compound 102 (or particles 104), for example.

In some embodiments, the chemical reaction described herein may comprise a reaction affecting the chemical properties of the adhesive compound 102 and/or particles 104, such as by any one or another, or one or more of the following: (i) decomposition—where a reactant is decomposed or broken down into two or more products, this may lead to digestion, for example; (ii) combination—where a plurality of reactants and reagents form a single product; (iii) substitution—where a single free element replaces or is substituted for one of the elements in a compound; (iv) metathesis—(e.g. ionic exchange) where two species are displaced, such as where the adhesive compound 102 and the particles 104 are ionic compounds, the positive ion in the adhesive compound 102 combines with the negative ion in the particles 104, and the positive ion in the particles 104 combines with the negative ion in the adhesive compound 102.

In some embodiments, the lubrication reaction described herein may comprise a reaction reducing friction within the adhesive compound matrix at the micro-cracks, which leads to the weakening and disintegration of the detachable adhesive composition 100.

According to some embodiments of the present disclosure, a mix for an adhesive is described. The mix which may be dry or liquid or part dry and part liquid includes a First Compound which is any of glue or resin or bond or cement material compound, any of which may be a composite mix of compounds itself , selected from various glue or resin or bond or cement compounds or compound mixtures, referred thereafter alternatively as Cement A, and a Second Reversible Compound, herein referred to as SRC or Compound B, mixed within the First Compound. When mixing the SRC with the first compound, particles of the SRC do not interact with and are not modified by the First Compound. Mix of SRC with the First compound is at certain weight or volume ratio, and if the first compound has particles, at certain relative ratio between the size of the SRC particles to those of the First Compound particles, or otherwise if Cement A is in liquid form, compound B particles has to have a certain maximal size for the mix to achieve the desired functionality. The mix forms together a Modified Reversible Adhesive herein referred to as MRC.

In some embodiment, the mix for an adhesive may comprise the detachable adhesive composition 100. The First Compound, namely cement A, may comprise the adhesive compound 102. The Second Reversible Compound, herein referred to as SRC or Compound B may comprise the particles 104.

The MRC can be used in fixing stage and removing stage at least once. In the fixing stage the MRC mix, is mixed together or exposed to or gets in contact with additional materials herein referred to as Hardening Materials used with the First Compound, and or used with other means herein referred to as Hardening Means, already used with the First Compound, to adhere the MRC mixture to a desired surface and or to itself and solidify. Said Hardening materials and Hardening means do not interact with and do not affect the SRC particles present in the MRC.

In the removing stage, when it is desirable to break the MRC adherence, the SRC is activated by external energy source, other than mechanical, to break the MRC adherence to a to a surface and or to itself permanently or reversibly. Thereafter, the MRC can be easily removed from or broken without application of significant mechanical effort or machinery.

The MRC mix may be premixed together, in various forms including in an applicator before use. Or the MRC may be mixed just on time together with the materials and methods applied to the First Compound.

When premixed in an applicator or other device, the MRC may already be in the fixing stage. The applicator or other device applies energy to the MRC in order move it to the removing stage just when it is needed to be used, following application of the MRC on the desired structure, the MRC return to the fixing stage.

Any adhesives or glue or resin or bond or cement materials compounds or compound mixtures and methods for hardening them, may be used as the First Compound material and Hardening Materials and Hardening Methods.

Device for applying energy to the hard MRC which can be any of thermal, light ultrasonic, microwave or other electromagnetic energy while in contact or without contact with the hard MRC is also described.

Many adhesives or glues or bonding materials or resins or cements are in use today in variety of applications. For clarity of description and without limitation we refer to them all as cements, cement materials or cement compounds or cement. In some cases, there are situations where it is desirable to break the cement to remove parts which the cement was used for. For non-limiting example, in dental treatment cement which is used in a cavity may be required to be removed to enable new treatment, or in construction cement that used to hold parts should be removed in renovation. The removal requires efforts machinery and time and might cause additional damage to the surroundings. It is therefore desirable to have a cement that is reversible and can be activated by applying energy source other than mechanical, by a device, to break the adherence without significant efforts while the cement adherence when not reversed is adequate for the purpose the cement is used for.

In some embodiments of the present disclosure a compound denoted herein as compound B is added at certain ratio to cements, materials and compounds, denoted herein without limitation as Cement A. Adding compound B may be done at various timing with respect to the use of the mixture, including premixing compound B with Cement A long before use of the mixture, or just at the time of use. If Cement A is made of particles, compound B particles size has certain ratio to the size of Cement A particles or otherwise if Cement A is in liquid form, compound B particles has to have a certain maximal size for the mix to achieve the desired functionality. The mixture of compound B with Cement A is referred to herein, as Modified Cement Mix (MCM).

In the fixing stage, materials and methods which are used on Cement A to adhere it to certain materials and or to itself, are applied on the MCM, to adhere it to the same certain materials Cement A adheres to, and/or to itself. For clarity and without limitation, after the fixing stage the MCM is referred herein as Fixed MCM or FMCM. Adding compound B to Cement A does not alter the original cement A adherence significantly. Removing stage is referred to herein as the time when after fixing of the MCM, it is desirable to remove the FMCM momentarily or permanently. In the removing stage, compound B present in the FMCM, may be activated by applying any of external energy sources which include without limitation: thermal (heat or cold), microwave, light or any other electromagnetic field, sonic or ultrasonic. Activation field may be applied by a device which is in contact with the FMCM or without physical contact with the FMCM. Activation alters the physical properties of compound B and causes fractions in the FMCM. Such fractions break the FMCM and allow easy removal of the activated FMCM.

In some embodiments, when application of activation field on compound B stops, compound B retains back its physical properties after time, and the activated FMCM may return back to be FMCM.

In some embodiments the process of fixing and activation for removal may be repeated several times on the MCM and or the FMCM.

In some embodiments, there is provided devices to apply the energy field in the removal stage which include means to control the energy source, including without limitation, type of energy, and other properties of the energy field, which may further include without limitation properties like amplitude or phase, number of pulses, pulse duration, and frequency.

In some embodiments there is provided devices to apply the MCM and or devices containing FMCM which may be used to apply activated FMCM that return to FMCM, following application.

As non-limiting example compound B may be Phase Change material (PCM) particles in form of solid powder particles which are not microencapsulated PCM, since use of microencapsulated PCM might not achieve the desired functionality. Furthermore, it might be that not all types of PCM may be suitable to achieve the desired functionality and only certain type of PCM may be used. If Cement A is made of particles, compound PCM particles size has certain ratio to the size of Cement A particles or otherwise if Cement A is in liquid form PCM particles has to have a certain maximal size for the mix to achieve the desired functionality. When PCM is exposed to temperature above the phase transition temperature of the PCM, it changes state from solid to liquid. PCM powder is first mixed with Cement A to form MCM. Mixing the PCM powder with Cement A is performed to achieve a distribution which may be a distribution as close as possible to uniform distribution of PCM particles within particles of Cement A. Then during use the MCM is first mixed or gets in contact with or exposed to hardening material (which is regularly used as hardener for Cement A) and the mixture is used as needed. Following hardening the PCM particles maintain their properties and are distributed within the FMCM. The distribution of PCM particles within the FMCM may be uniform distribution or any other distribution as may be desired. Such FMCM has similar properties to those of Cement A following hardening, and therefore it is good for use in the same application Cement A is used for. If and when needed to be removed, activation of the FMCM is performed by applying a thermal energy source at the vicinity of the FMCM moving the FMCM to a temperature above the phase transition of the PCM. Then, the PCM particles embedded in the FMCM melt and expand, this in turn apply force on the FMCM which breaks it. To stop the activation, the thermal energy source should be stopped. Sometime after the activation stops, the PCM particles return to their solid state and for some types of activated FMCM they return to be hard FMCM again. It is also possible to control such process and time by adjusting the thermal energy source to apply temperature above and PCM phase transition to activate the FMCM and then below the temperature of the phase transition of the PCM in order to harden the activated FMCM back again.

In yet another non-limiting example compound B may be made of microencapsulated water. The FMCM in this non-limiting example may be activated by applying any of thermal, light or microwave field on the FMCM. Activation, in this non-limiting example causes the microencapsulated water to boil, evaporate or ablate and in turn breaks the FMCM.

Other microencapsulated materials or particles may be used as compound B instead of microencapsulated water. Such particles or microencapsulated materials are not affected by the Hardening Materials and Hardening Methods and do not interact with Cement A. The energy source type used for activation is adjusted in the activation device according to the type of compound B used in the MCM. This energy can be any thermal, light or microwave magnetic or electromagnetic , sonic or ultrasonic.

In yet another non-limiting example compound B may made of any type of water absorbent granular material such as described in U.S. Pat. No. 6,067,806 patent. The FMCM in this non-limiting example may be activated by applying any of thermal, light or microwave magnetic or electromagnetic, sonic or ultrasonic field on the FMCM. Activation, in this non-limiting example causes the water contained within the particles of compound B to boil, evaporate or ablate and in turn breaks the FMCM

In yet another non-limiting example compound B may made of any type of particles made of micro shells comprising material wherein when FMCM is activated the micro shells expand and break thereby releasing the material comprised inside. The material interacts with surrounding to break the adhesion The FMCM in this non-limiting example may be activated by applying any of thermal, light or microwave magnetic or electromagnetic field or sonic or ultrasonic field on the FMCM.

In another preferred embodiment application of the energy source to activate the FMCM is through a layer of buffer material (i.e. like liquid) while the device applying the field does not come in direct contact with the surface of the material to which the FMCM is adhered. As non-limiting example in case of FMCM used in dental applications, when needed the moth of the patient is filled with water and the tool applying the energy source is inserted to the moth to emit energy all around without need to contact a specific tooth, reacts only with the particles of material B embedder in FMCM wherever FMCM is present, and break it.

In another preferred embodiment only upon the application of combination fields (constant or variable) particles of B embedded in FMCM react to change their physical state and physically or chemically or physically and chemically react with the hard cement to cause it to break in a reversible or non reversible manner.

PCM material with melting temperature of 108° C. or 151° C. like PureTemp 108 or PureTemp 151 from Entropy Solutions may be grind to powder forming compound B. Thereafter, compound B may be mixed with cement like Portland Cement or any other type of cement used in construction at weight ratio of 1:10 or 1:5 or 1:2 or volume ration of 1:2 or other ratios depending on the application to form MCM. For fixing this MCM is then mixed with water at certain desired ratios of water to MCM. Following mixing with water and before the mixture is fixed, the mixture is applied on surface like breaks or other surfaces to join them together after fixing. Following water evaporation and drying the MCM is hardened to FMCM form. To activate the FMCM a heating device is used. The heating device applies heat to increase the temperature of the FMCM to be greater than 108° C. or greater than 151° C., at a certain area where it is required to activate the FMCM for reconstruction purposes. The heating device should apply the heat for certain duration of time to achieve this. Such time can be 60 seconds or 120 seconds or 5 minutes or 10 minutes or any such time as may be needed to raise the temperature of the FMCM above 108° C. or 151° C. The device applying the heat does not need to be in contact with the surface containing the FMCM. Following such time as needed, the PCM undergoes phase transition and the FMCM is activated which allows removal of the surface containing the FMCM and or the FMCM from the surface it was used on. This is achieved without need to use mechanical machinery for the task.

Various types of PCM's having various phase transition temperatures may be used as compound B, and mixed together with other kinds of cements like Acrylic based, Polymer or Epoxy based cement materials used as Cement A. More than one type of compound B may be used in a mixture together with Cement A. following fixing, by applying proper temperature on various areas ,a profile of activation of the FMCM may achieved , allowing specific areas to be activated according to the desired response and requirement.

The PCM type and its phase change temperature may be selected according to the anticipated use of the FMCM. Such that under normal use of the surface with the FMCM it will not get to the phase change temperature and only application of heat by a dedicated device will get the PCM to undergo phase transition.

PCM powder may be added to any type of the materials or similar materials used in construction similar to the materials available from http://www.carmit-mrfix.com . PCM should be made in powder form to uniformly spread the PCM particles over the volume of the cement

The PCM in the above examples may be replaced with microencapsulated water or other materials which are microencapsulated forming compound B. Activation of the FMCM is achieved by adjustment of the energy source properties applied by the activation device on the FMCM or the surface in contact with the FMCM according to the application

In a non-limiting example for medical dental application, PCM as powder particles, or microencapsulated water or other material is microencapsulated forming compound B. Compound B is mixed with materials used in medical dental applications such as BC SEALER from BRASSELER USA, Harvard Cement, RelayX Unicem by 3M, GC Fuji IX, Durelon, or any type of Glass Ionomer Cement (GIC) or other types of Cement A material used as cements in dental applications, to form MCM. During first treatment MCM is used and when retreatment is needed, the FMCM can be easily removed without using mechanical machinery by using the device which applies the energy required to activate the FMCM even without direct contact with the surface containing the FMCM.

Cement mixture which is made by mixing compound or material B particles which are in a first state with cement mixtures or cement compounds referred collectively as material A. The mixture is hardened by adding or exposing it to hardener material C which does not affect material B particles when they are in their first state but reacting with material A and forming hard mix having distribution of material B particles which are in a first state embedded within, and further wherein; Application of at least one energy source fields or combination of fields on the hard mix which may react only with material B particles embedded within, further causes material B particles embedded within to change their physical properties, move to a second state react physically and/or chemically with the hard mix and break the hard mix in reversible (i.e. become hard mix again) or non-reversible manner. In some embodiments, material B particles are of the same size or of size smaller than particles of Material A or of size significantly smaller ( 1/10) than those of material A. In some embodiments, material B particles are larger than particles of Material A or of size significantly larger (×10) than those of material A. In some embodiments, the combination of fields is of the same type or different type of fields (i.e sonic and electromagnetic). In some embodiments, at least one of the at least energy field is constant or varying in any of the fields characteristics. In some embodiments, at application of the at least one energy source field causes alternating physical reactions within the material B particles. In some embodiments, material B particles are mixed with cement mixtures or compounds or glue compounds or glue mixtures or bond compounds or bond mixtures or resin compounds or resin mixtures. Including without limitation Epoxies, Cements, Cyanoacrylates, spray adhesives, pressure sensitive adhesive, fabric adhesives, hot glue, Yellow wood glue, Polyurethane adhesive, Synthetic polymer or synthetic monomer, solvent type, plant based, animal based, mineral based, Portland Cement, BC Sealer. In some embodiments, material B particles are uniformly mixed with cement mixtures or compounds. In some embodiments, compound or material B is any of PCM powder particles or microencapsulated material. In some embodiments the cement or compound mixture which is mixed together with material or compound B is any of cements compounds used in construction or cements used in medical dental applications. In some embodiments, the compound or material B is in liquid or solid particles form. In some embodiments, the cement or compound mixture which is mixed together with material or compound B is in liquid or solid particles form. In some embodiments, the cement mixture is premixed together in an application device containing material which following mixing with the mixture causes it to become hard. Method to apply energy to a hard-solid mixture of particles having distribution which may be uniform, of solid particles of one material among hard other material or hard mix of other materials, wherein the particles of the one material absorb the energy and change their physical properties, causing the hard-solid mixture to break in reversible or non-reversible manner. Device to apply energy to a hard-solid mixture of particles having distribution which may be uniform, of solid particles of one material among hard other material or hard mix of other materials, wherein the particles of the one material absorb the energy and change their physical properties. Causing the hard-solid mixture to break in reversible or non-reversible manner. In the device the application of the energy to the hard mix may be with direct physical contact with the material or without physical contact with the material.

While various inventive embodiments have been described and illustrated herein, those of ordinary skill in the art will readily envision a variety of other means, materials, or structure for performing the function, obtaining the results, or one or more of the advantages described herein, and each of such variations or modifications is deemed to be within the scope of the inventive embodiments described herein. More generally, those skilled in the art will readily appreciate that all parameters, dimensions, materials, and configurations described herein are meant to be for example only and that the actual parameters, dimensions, materials, and configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims, equivalents thereto, and any claims supported by the present disclosure, inventive embodiments may be practiced otherwise than as specifically described and claimed. Inventive embodiments of the present disclosure are directed to each individual feature, system, article, material, composition, kit, method, and step, described herein. In addition, any combination of two or more such features, systems, articles, materials, compositions, kits, methods, and steps, if such features, systems, articles, materials, compositions, kits, methods, and steps, are not mutually inconsistent, is included within the inventive scope of the present disclosure. Embodiments disclosed herein may also be combined with one or more features, functionality, or materials, as well as complete systems, devices or methods, to yield yet other embodiments and inventions. Moreover, some embodiments, may be distinguishable from the prior art by specifically lacking one and/or another feature disclosed in the particular prior art reference(s); i.e., claims to some embodiments may be distinguishable from the prior art by including one or more negative limitations.

Also, as noted, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A detachable adhesive composition for selectively adhering to an object and detaching therefrom. comprising:
an adhesive compound; and
a plurality of selectively activated particles,
wherein:
the composition is configured to adhere to an object when the selectively activated particles are inactive; and
the plurality of selectively activated particles are configured to be activated upon exposure to and absorption of energy causing the selectively activated particles to flow and expand so as to form micro-cracks in the adhesive compound, flow into the micro-cracks, and react with the adhesive compound by at least one of:
at least partially chemically reacting with adhesive compound so as to at least partially digest the adhesive compound, and
lubricating the adhesive compound so as to allow the detachable adhesive composition to detach from the object.

2. The composition of claim 1, wherein the selectively activated particles are selectively flowable.

3. The composition of claim 1, wherein the composition selectively adheres an article to the object.

4. The composition of claim 1, wherein the composition is configured for selectively adhering to the object for filling cavities within the object.

5. The composition of claim 3, wherein the article comprises at least one of a dental crown, bracket, bridge or brace, and the object comprises any one of a tooth, dental implant or abutment.

6. The composition of claim 1, wherein the composition forms a gel nail polish, and the object comprises a human nail.

7. The composition of claim 3, wherein the article comprises at least one of a tile or flooring, and the object comprises any one of a floor, wall or ceiling.

8. The composition of claim 1, wherein the plurality of selectively activated particles comprise a weight or volume of 1% to 10% of a total weight or volume of the composition.

9. The composition of claim 1, wherein:
the adhesive compound is configured with a first level of a reacting parameter (RP), the selectively activated particles are configured with a second level of RP, and the difference between the first level the second level is configured to be:
at a first predetermined value, whereupon the selectively activated particles are at least partially inactive and thereby nonreactive with the adhesive compound, and
at a second predetermined value whereupon the selectively activated particles are activated.

10. The composition of claim 9, wherein the RP comprises a pH level.

11. The composition of claim 9, wherein each level of RP is measured by RP units and the second predetermined value is greater than an RP unit of 5.

12. The composition of claim 1, wherein the adhesive compound includes an original of less than 7 and the plurality of selectively activated particles includes an original pH of greater than 7.

13. The composition of claim 1, wherein the adhesive compound comprises at least one of a cement, glue or binder.

14. The composition of claim 1, wherein the selectively activated particles comprise a phase change material.

15. The composition of claim 1, wherein the selectively activated particles comprise granules each having a diameter of less than 300 microns.

16. The composition of claim 1, wherein the plurality of selectively activated particles comprise granules each having a diameter of less than 100 microns.

17. The composition of claim 1, wherein the plurality of selectively activated particles comprise granules each having a diameter of less than 50 microns.

18. The composition of claim 1, wherein the plurality of selectively activated particles comprise an un-encapsulated phase change material.

19. The composition of claim 1, wherein the plurality of selectively activated particles comprise microencapsulated water.

20. The composition of claim 1, wherein:
the plurality of selectively activated particles comprise a microcapsule
comprising an encapsulated material, and
the microcapsule is formed of a material configured to burst upon the application of the energy to facilitate the flow of the encapsulated material into the adhesive compound.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)
